(19) United States
(12) Patent Application Publication
(10) Pub. No.: US 2022/0133391 A1
(43) Pub. Date: May 5, 2022

(54) ELECTROSURGICAL GENERATOR WITH INVERTER FOR GENERATING HF HIGH VOLTAGE

(71) Applicant: OLYMPUS WINTER & IBE GMBH, Hamburg (DE)

(72) Inventors: Jelle DIJKSTRA, Berlin (DE); Stefan SCHIDDEL, Stahnsdorf (DE); Thomas PREZEWOWSKY, Teltow (DE)

(73) Assignee: OLYMPUS WINTER & IBE GMBH, Hamburg (DE)

(21) Appl. No.: 17/516,122

(22) Filed: Nov. 1, 2021

(30) Foreign Application Priority Data
Oct. 30, 2020 (DE) 10 2020 128 589.2

(51) Int. Cl. A61B 18/12

(52) U.S. Cl. CPC A61B 18/1233; A61B 2018/1226

(57) ABSTRACT

An electrosurgical generator includes a power supply unit which, when operating, supplies a direct voltage circuit, and a high-voltage inverter supplied from it that generates a high-frequency alternating voltage that is applied to outputs for connection of the electrosurgical instrument. The inverter includes a clock-driven power switch and a zero-crossing detector that recognizes zero crossings of the oscillation generated by the inverter. A signal for the generated alternating voltage is applied to the zero-crossing detector via a voltage divider which is a capacitive voltage divider with at least one capacitor that is resistant to high voltage. Undesirable direct voltage components at the center tap in the presence of changes to the supply voltage can be avoided thereby, since charge reversals as a result of changes to the supply voltage occur on both sides, and their effects thus cancel out.

The invention relates to an electrosurgical generator that is designed to output a high-frequency alternating voltage to an electrosurgical instrument. It comprises a power supply unit which, when operating, supplies a direct voltage circuit, and an inverter for high voltage that is fed by the direct voltage circuit and generates a high-frequency alternating voltage that is applied to outputs for connection of the electrosurgical instrument.

High-frequency alternating current is used in electrosurgery in particular for cutting or separating tissues and for the removal of bodily tissue in the sense of thermal resectioning (known as an electrical scalpel). The functioning principle is based on the heating of the tissue that is to be cut. One advantage of this is that bleeding can be stopped by sealing the affected vessels at the same time as the incision. Not inconsiderable powers are required for this, and these must be provided at frequencies of 100 kHz or more, up to 4000 kHz, typically around 400 kHz. At these frequencies, bodily tissue behaves like an ohmic resistor. The resistivity, however, greatly depends on the type of tissue, and the resistivities of muscles, fat or bone differ greatly from one another, specifically by up to a factor of 1000. The result of this is that in operation the load impedance of the electrical scalpel can change strongly and rapidly, depending on the tissue to be cut. This places specific and characteristic demands on the electrosurgical generator, and in particular on its inverter. Rapid voltage regulation is necessary in particular in an environment with high voltages in the range from a few kilovolts and of a high frequency in the range of typically between 100 kHz and 4 MHz.

To meet these unique requirements, electrosurgical generators are typically constructed such that they comprise an inverter to supply power to the electrosurgical instrument to which rectified current from the mains is supplied at varying voltages. This rectified current is provided from the mains by a high-voltage DC power supply (or high-voltage power supply—HVPS). The inverter, in turn, is typically implemented as a free-running single-ended generator. To generate and maintain the oscillation, this needs the zero-crossing of the generated oscillation to be ascertained. Due to the high voltage level in the generators, with peak voltages of up to 1000 volts, it is necessary for this value to be divided down to a lower voltage level suitable for the further processing and detection. A voltage divider consisting of a capacitor resistant to high voltage and a resistor is usually used for this purpose.

A particular difficulty results from the fact that the output power of the electrosurgical generator is controlled by the supply voltage. As a consequence, the direct voltage component of the generator output voltage also changes with every change in the supply voltage. This leads to incorrect detections of the zero crossing, because the capacitor of the voltage divider undergoes a charge reversal whenever the supply voltage changes. Incorrect interpretations of this sort can result in incorrect switching of the power semiconductors in the inverter or to stalling of the oscillation. To avoid this, the rise and fall rates of the generator supply voltage must be limited, and this entails the risk that sufficiently rapid adjustment cannot occur when the load impedance changes quickly. This is a significant disadvantage for the operational safety and for the quality of the supply of the electrosurgical instrument.

The invention is based on the object of improving an electrosurgical generator of the type mentioned at the beginning in such a way that its operating behavior becomes more robust, particularly in respect of the detection of zero crossings.

The solution according to the invention is found in the features of the independent claim. Advantageous developments are the subject matter of the dependent claims.

In an electrosurgical generator designed to output a high-frequency alternating voltage to an electrosurgical instrument, comprising a power supply unit which, when operating, feeds a direct voltage circuit, and an inverter for high voltage that is fed from the direct voltage circuit and generates a high-frequency alternating voltage that is applied to outputs for connection of the electrosurgical instrument, wherein the inverter has a clock-driven power switch and a zero-crossing detector designed to detect zero crossings of the oscillation generated by the inverter, wherein a signal for the generated alternating voltage is applied to the zero-crossing detector by means of a first voltage divider via a signal line, it is provided according to the invention that the voltage divider is designed as a capacitive voltage divider for alternating voltage with at least one capacitor that is resistant to high voltage.

The core of the invention is that with a capacitive voltage divider in which capacitors are used on both sides of the center tap, the occurrence of unwanted changes in the direct voltage component can be avoided at the center tap when supply voltage changes occur. This is based on the recognition that when capacitors are provided toward both the higher and the lower potential, the charge reversals that occur when the supply voltage changes happen symmetrically, and thus cancel out the effects of the charge reversals. This cannot be achieved in a conventional voltage divider. The desired blocking of the direct voltage cannot be achieved in a conventional resistor/resistor voltage divider. While blocking of the direct voltage can be achieved by means of the RC voltage divider frequently used in the prior art, the charge reversal of the capacitor, present only on one side, that occurs when the supply voltage changes leads to a direct current component in the divided voltage, which then leads to the disadvantages mentioned at the beginning. The design according to the invention, using a purely capacitive voltage divider, avoids this in a strikingly simple way. This has the further advantage that its division ratio is frequency-independent, unlike that of a conventional RC voltage divider.

The capacitive voltage divider further offers the advantage of reliable detection even at small output voltages, as typically occur when only low output powers of a few watts are demanded by the electrosurgical generator. The oscillation then frequently stalls in the generators used in the prior art. This can be avoided with the capacitive voltage divider according to the invention, since the zero crossings can be detected more accurately and more quickly. The quality of detection is thus noticeably improved. Overall, greater operational safety thus results, in particular also in respect of large changes to the supply voltage, and in relation to large jumps in the load impedance, which benefits robust functional safety of the generator and thus, in the end, also the safety of the patient.

It is, furthermore, sufficient if at least one of the capacitors of the capacitive voltage divider is resistant to high voltage.

Overall, significant advantages in respect of operational safety, robustness and, finally, patient safety, can thus be achieved with what at first sight appears as a surprisingly simple measure.

A few terms used should first be explained below:

In the field of electrosurgical generators, “high frequency” refers to frequencies typically in the range between 100 kHz and 4000 kHz. “High voltage” typically refers to voltages up to 10 kV, preferably up to 4000 V.

Supply voltage refers to the voltage that is present in the direct voltage circuit.

The term of a signal for the generated alternating voltage includes, in particular, signals for the magnitude, frequency, phase position and/or amplitude of the generated alternating voltage.

The power provided by the electrosurgical generator typically lies in the range between 1 and 500 watts, wherein the load impedance can vary greatly, and the output voltage and power output can accordingly change equally greatly and suddenly.

In the present case, the term of the “zero-crossing detector” is to be understood broadly, and also comprises detectors in which the threshold to be detected is not exactly at zero, but can be shifted by means of a reference.

The capacitive voltage divider preferably has a division ratio between 1:20 and 1:4. The division ratio is defined by the ratio of the capacitance of the upper capacitor Cₒ to that of the lower capacitor Cᵤ, wherein the output voltage Uₐ across the lower capacitor Cᵤ is defined as

$$U a = \frac{C o}{C o + C u} \cdot Ue$$

of the total input voltage Uₑ present across the two capacitors. The capacitive voltage divider must be dimensioned here in such a way that two opposing goals are reached. The high voltage must first be divided down sufficiently far that it can be processed by the subsequent electronics, which are not resistant to high voltage; on the other hand, it must not be divided down too far, so that sufficiently large voltage signals can be obtained by the voltage divider even when the supply voltage is low (for example when the power requirement is low or the load is of exceptionally low impedance). A ratio of 1:6 has been found particularly effective.

It is expedient for the capacitors of the capacitive voltage divider to have values in the range between 50 pF and 10 nF. Impedances X_{C} in the range between 80 and 16 kOhm thus result at a typical frequency of 200 kHz.

The capacitive voltage divider is advantageously arranged in parallel with the power switch. The input voltage of the capacitive voltage divider thus corresponds to the voltage dropped across the power switch, in particular a power MOSFET.

The capacitive voltage divider can optionally be connected directly, immediately to the alternating voltage generated by the power switch; it is, however, preferable if it is not connected directly to the alternating voltage generated by the power switch, but rather by way of a current-limiting element. In this way, any current peaks that may occur in the capacitive voltage divider can be avoided or limited. The current-limiting element is expediently implemented as a low-ohm resistor, the resistance value of which is smaller, preferably at least an order of magnitude smaller, than the impedance of the capacitive voltage divider. The result is thus that in this way there is only a negligibly small effect on the capacitive voltage divider, but an effective current limitation nevertheless results.

The signal line advantageously comprises a correction circuit that is designed to minimize or remove a direct voltage potential in the signal line. In this way, the possibility that a direct voltage potential might develop at the output of the capacitive voltage divider or in the signal line can be prevented. Such an unwanted direct voltage potential would have a disturbing effect on the input of the following zero-crossing detector. This can be effectively prevented with the correction circuit. A particularly simple but nevertheless efficient circuit for overcoming the direct voltage offset is found in a resistor, preferably in the range of kilohms, connected to ground. The resistor is expediently dimensioned such that, taking the capacitances in the capacitive voltage divider into consideration, a high-pass filter is created with a 3 dB cut-off frequency below the high-frequency range of the generator. There is therefore no negative effect from the correction circuit for the high-frequency range used by the electrosurgical generator.

According to a particularly preferred embodiment, which may merit independent protection, a variable reference is applied, preferably via a reference line, to the zero-crossing detector as a zero reference. The zero threshold of the zero-crossing detector can be raised with the variable reference. The detection threshold for ascertaining the zero crossing can thus be shifted upwards, which also enables correct detection of the zero crossing even with pulsed and heavily damped voltages at the generator output. The risk of detecting what might be called “false” zero crossings is thereby minimized. Overall, this therefore allows zero crossings of the alternating voltage to be detected more accurately and more quickly. This benefits the robustness of the detection, and thereby the operational safety of the generator as a whole, in particular in respect of tolerance to large jumps in the load impedance.

The variable reference is advantageously derived from the voltage in the direct voltage circuit. In this way, the detection threshold is raised together with rising supply voltage, so that even with a load-dependent decay of the inverter, as can in particular occur with critical damping at the generator output with certain load impedances, the zero crossings continue to be correctly detected.

The variable reference is expediently generated by means of a second voltage divider, and this may be done from the voltage in the direct voltage circuit. The second voltage divider is preferably constructed in a different way, in particular resistively, as compared with the first (capacitive) voltage divider. Its division ratio is expediently smaller than that of the capacitive voltage divider, preferably being between one fifth and one tenth. An impedance converter is advantageously connected between the second voltage divider and the zero-crossing detector, preferably being implemented as a buffer amplifier. This ensures that the reference input to the zero-crossing detector is decoupled from the second voltage divider, and the second voltage divider is thus not unnecessarily loaded, which otherwise could lead to an undesirable distortion of its output signal.

An offset circuit can furthermore advantageously be provided in the reference line, designed, in the absence of an input signal from the second voltage divider, to apply a defined reference, preferably other than zero, to the zero-crossing detector via the reference line. Due to this offset circuit, a certain voltage, typically a low positive voltage, is always present at the reference input to the zero-crossing detector. This initial voltage has the result that if a measurement signal from the capacitive voltage divider is (still) missing, i.e., if the value in the signal line is zero, the zero-crossing detector always adopts a defined position and accordingly outputs a defined output signal. To avoid unnecessary effort, the offset circuit is expediently integrated into the impedance converter, preferably as a pull-up resistor or pulldown resistor. Since a reference that differs from zero is applied to the zero-crossing detector, the offset circuit makes it possible to prevent an undefined state from occurring at the buffer amplifier and consequently at the zero-crossing detector.

It is furthermore expedient if limiting circuits are provided at the input to the zero-crossing detector in the signal line and/or the reference line, preferably comprising protective diodes connected antiparallel and/or a high-pass filter. An effective voltage limitation and protection against harmful consequences of harmonic components in the signal line can be achieved in this way at the zero-crossing detector, which on the one hand provides protection to the components, and on the other hand increases the operational safety.

The invention is explained in more detail below with reference to an advantageous exemplary embodiment. In the figures:

FIG. 1 shows an electrosurgical generator according to one exemplary embodiment with an attached electrosurgical instrument;

FIG. 2 shows a schematic functional diagram of the electrosurgical generator according to FIG. 1;

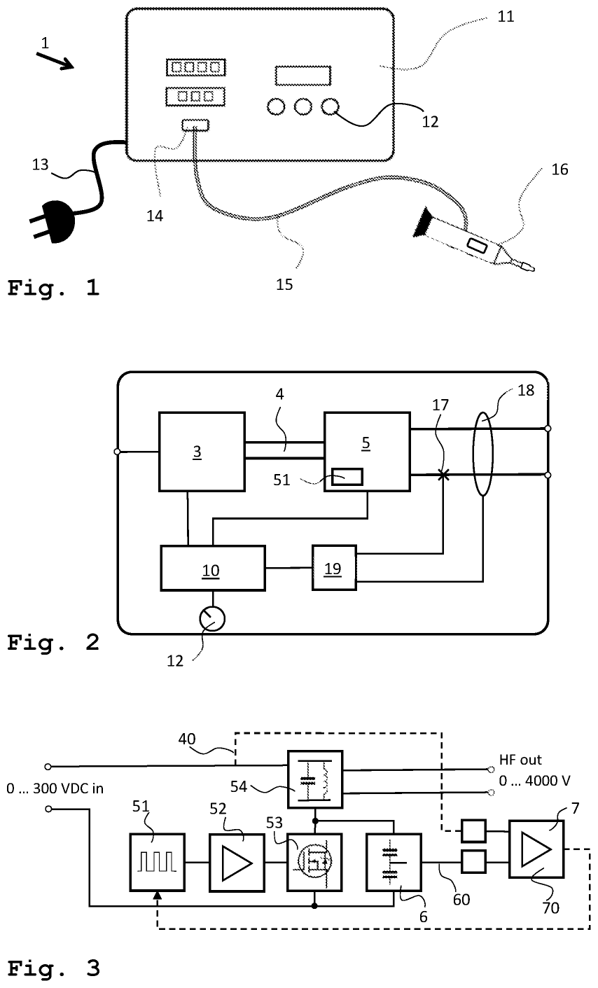

FIG. 3 shows a block diagram of an inverter of the electrosurgical generator according to FIG. 1;

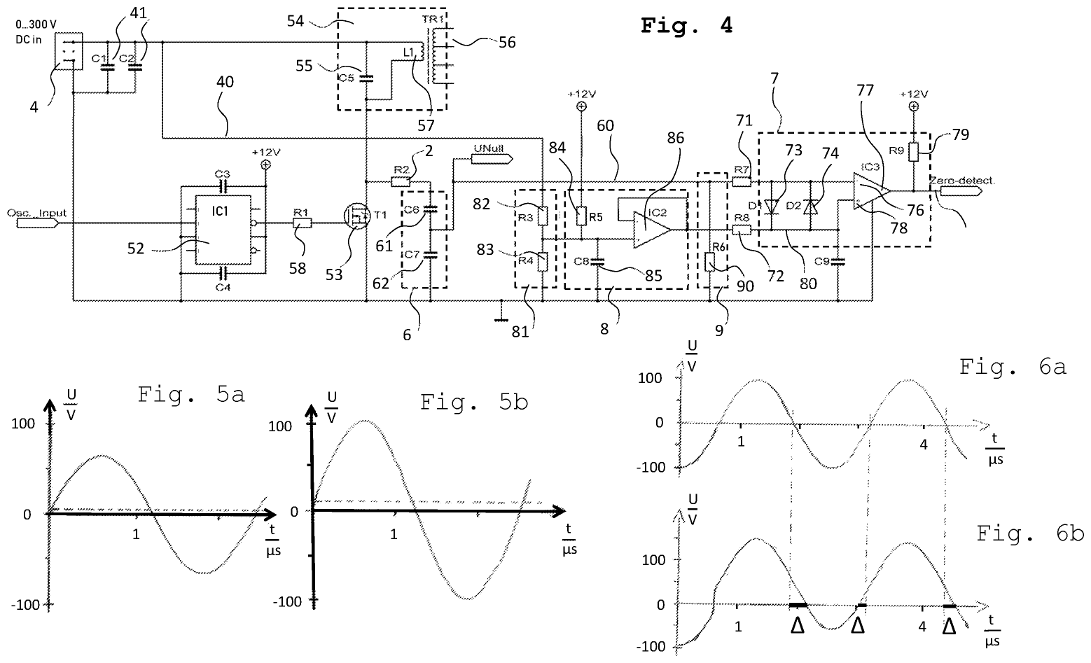

FIG. 4 shows an exemplary circuit diagram of the inverter with power switch and zero-crossing detector;

FIGS. 5a, b show graphs of voltage curves;

FIGS. 6a, b show graphs of voltage curves and zero crossings according to the prior art; and

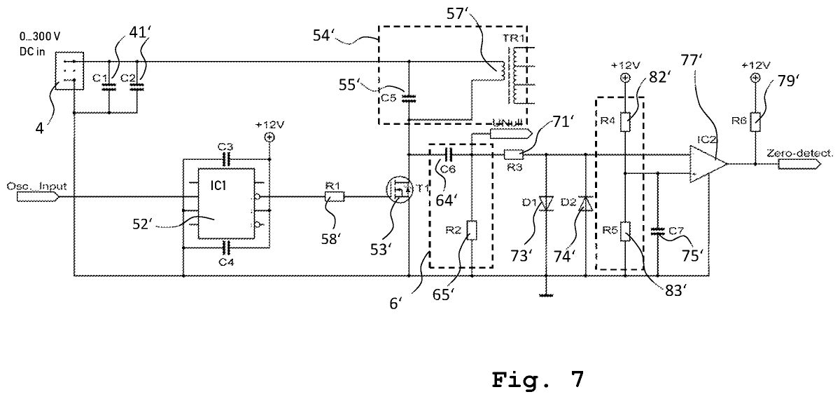

FIG. 7 shows a circuit diagram of an inverter according to the prior art.

An electrosurgical generator according to one exemplary embodiment of the invention is illustrated in FIG. 1. The electrosurgical generator, identified as a whole with reference sign 1, comprises a housing 11 provided with a terminal 14 for an electrosurgical instrument 16 which, in the exemplary embodiment illustrated, is an electrical scalpel. It is connected via a high-voltage connecting cable 15 to the terminal 14 of the electrosurgical generator 1. The power output to the electrosurgical instrument 16 can be changed by means of a power controller 12. A mains connecting cable 13, which can be connected to the public electricity mains, is provided for the supply of electrical power to the electrosurgical generator 1.

A schematic functional diagram of the electrosurgical generator 1 is illustrated in FIG. 2. It comprises a power supply unit 3 that is supplied with electrical power by the mains connecting cable 13 (see FIG. 1). The power supply unit 3 is a high-voltage power supply unit (HVPS). It comprises a rectifier and feeds a DC link 4 with direct voltage, the level of which can vary between 0 and about 300 volts in the embodiment illustrated, wherein the absolute level of the direct voltage depends in particular on the set power, the type of electrosurgical instrument 16 and/or its load impedance, which in turn depends on the type of tissue being treated.

An inverter 5 that generates high-frequency alternating current in the high-voltage range of a few kilovolts is fed from the DC link 4. The inverter 5 is of the type with a free-running single-ended generator. The high-frequency high voltage output at the terminal 14 is measured by means of voltage and current sensors 17, 18, and the measurement signals are supplied to a processing unit 19 that applies the corresponding data regarding the voltage, current and power that are output to an operating controller 10 of the electrosurgical generator 1 to which the power controller 12 is also connected.

In a free-running single-ended generator, as is typically used in the inverter 5 for electrosurgical generators 1, it is necessary for the sake of stable operation that the zero crossing of the oscillation generated is detected correctly. For this purpose, a zero-crossing detector 7 is provided which makes a signal for the zero crossing available at its output via a line 70 which is applied to an oscillation control unit 51.

This is illustrated in more detail in FIG. 3, which shows a block diagram of the inverter 5 with its power stage. A parallel resonant circuit 54 comprises a high-voltage capacitor 55 and an inductor 57 that is preferably the primary winding of a transformer 56 whose secondary side is connected to the output terminal 14. The upper terminal of the parallel resonant circuit 54 is connected to the upper potential of the direct voltage circuit 4, while its lower terminal is connected via a power switch 53 to the lower potential of the direct voltage circuit 4. The semiconductor power switch 53 is clock-driven by an oscillation control unit 51 via a driver 52 for decoupling and amplification. The power switch 53 is a power semiconductor, particularly of the MOSFET type, although other types of fast-switching power semiconductors may also be used. Through the fast, periodically clocked driving of the power switch 53, a corresponding alternating voltage is generated across the capacitor 54, which is then output via the transformer 56 at the terminal 14 as a high-frequency high voltage. The frequency of the periodically clocked driving can be changed and is largely determined by the parallel resonant circuit 54.

To detect the zero crossings, the voltage at the drain terminal of the power switch 53, i.e., at the connection between the power switch 53 and the parallel resonant circuit 54, is tapped off by means of a voltage divider 6.

Before the embodiment according to the invention is explained in more detail, reference will first be made to the implementation of this topology according to the prior art, as is illustrated in the circuit diagram according to FIG. 7. The input for the supply voltage from the direct current circuit 4, together with smoothing capacitors 41′, can be seen at the top left. The input for the clocked oscillation signal that acts on the driver 52′, which in turn drives the power switch 53′ via a protective resistor 58′, can also be seen at the left-hand edge. This is connected to the resonant circuit 54′ which comprises a capacitor 55′ and an inductor 57′. A voltage divider 6′ is connected to the drain terminal of the power semiconductor 53′, in order to tap off the voltage for detection of the zero crossing. The voltage divider 6′ is formed by a high-pass filter with a capacitor 64′ that is connected to the drain terminal of the power switch 53′, and a resistor 65′ that connects the capacitor 64′ to the lower potential of the direct voltage circuit. At its output, the voltage divider 6′ outputs the voltage U_{Null} which is output via a voltage limiting circuit comprising a resistor 71′ and diodes 73′, 74′ connected antiparallel, and is applied to a negative input of the comparator 77′ that acts as the zero-crossing detector. A second voltage divider 81′ with the two resistors 82′, 83′ is connected to the other, positive input of the comparator 77′. They form the zero reference against which the comparator 77′ compares the voltage signal measured by the voltage divider 6′. The resistors 82′ and 83′ are dimensioned in the exemplary embodiment illustrated in such a way that a small, positive offset voltage, which is thus not exactly at zero, results. In this way, it is ensured that, even in the absence of a signal from the voltage divider 6′, the comparator 77′ always outputs a defined signal, namely a positive output voltage, and an undefined state therefore cannot arise. A pull-up resistor 79′ is provided there, again to avoid undefined states at the output of the comparator 77′. In regular operation, when a high-frequency signal is generated by the electrosurgical generator 1 (typically in the range between 300 and 600 kHz) the output of the comparator 77′ continuously changes in time with the voltage at the output of the comparator 77′ tapped off by the voltage divider 6′, between 0 V when the voltage U_{Null} present at the negative input exceeds the reference set by the voltage divider 81′ and a positive output voltage when the voltage U_{Null} falls below the set reference. In this way, in the settled state, the zero crossing of the alternating voltage generated by the electrosurgical generator can be detected and processed further.

As already explained at the outset, the disadvantage of this circuit is relevant in particular when the voltage with which the inverter is supplied is changed. This can happen intentionally by adjusting the power controller 12, but also through what may be a very fast change in the load impedance. If the supply voltage in the direct voltage circuit 4 changes, then the direct voltage component of the generated alternating voltage, as is also present at the voltage divider 6′, necessarily also changes. The result of this is that with each change in the supply voltage, the capacitor 64′ in the voltage divider 6′ is charged up or discharged in accordance with the changed direct voltage component, and this charge compensation leads to a direct current component. This additional direct current component leads to faulty detection of the zero crossing, which can then consequently lead to the oscillation stalling and/or to an incorrect switching of the power switch.

This is shown visually in FIG. 6. The regular settled state, in which the zero crossings are correctly detected at regular intervals, is illustrated in FIG. 6a. FIG. 6b shows that the supply voltage is increased as the oscillation continues. As a result of the direct component from the charge reversal of the capacitor, the alternating voltage curve, unchanged in itself, now rises to a higher potential, which has the consequence of a significant shift in the zero crossings. In FIG. 6b this shift can be seen in the discrepancy A between the vertical dashed line indicating the zero crossing time that is, in itself, correct, and the actual zero crossing time of the solid curve, which differs from it significantly. It can be seen straight away that the detection is significantly distorted.

The improved version according to the invention is described with reference to the circuit diagram of FIG. 4. The voltage supply and the driver 52 in the left-hand region of the circuit diagram, including the power switch 53 and the parallel resonant circuit 54, are as described above for FIG. 7. A different voltage divider is provided according to the invention, namely a capacitive voltage divider 6 that contains two capacitors 61, 62 that are resistant to high voltage. To protect them from any current peaks that may occur, the connection to the power switch 53 is made via a current limiting element 2, which, in the exemplary embodiment illustrated, is realized as a low-ohm resistor (in the range between 2 and 20 ohms). Due to this low resistance, influence on the capacitive voltage divider is extremely small, and can be disregarded. In the exemplary embodiment illustrated, the capacitors 61, 62 are dimensioned such that a division ratio of 1 to 6 results, i.e., the voltage across the power switch 53 is divided down by the voltage divider 6 to one-sixth of the value. This voltage signal is transmitted via a signal line 60 from the capacitive voltage divider 6 to the zero-crossing detector 7 or, put more precisely, to a negative input 76 of a comparator 77 of the zero-crossing detector 7.

A limiting circuit for the magnitude of the signal is provided along the signal line 60. It is realized by a series resistor 71 and two diodes 74, 75 arranged antiparallel between the signal line 60 and a reference line 80.

In respect of the reference signal transmitted on the reference line 80 to the comparator 77 of the zero-crossing detector 7, it is provided according to a particularly advantageous optional aspect of the invention that this is not fixed but is derived in a variable manner from the supply voltage. A second voltage divider 81, comprising two resistors 82, 83, is provided for this purpose. A measuring line 40 that applies the upper potential of the direct voltage circuit 4 to the upper terminal of the second voltage divider 81 is provided for this. Its lower terminal is connected to ground, and thus to the lower potential of the direct voltage circuit. In this way, a reference that follows the voltage level in the direct voltage circuit 4, and is therefore variable, can be generated. It is passed via an impedance converter 8 with a buffer amplifier 86 that is configured as a voltage follower. The voltage signal tapped off from the second voltage divider 81 is applied to the positive input of the buffer amplifier 86, while a pull-up resistor 84 and a capacitor 85 are furthermore provided to improve the signal. The pull-up resistor 84 ensures a positive initial voltage even when no measurement signal is transmitted from the second voltage divider 81. Feedback from the output is connected in the manner known per se to the negative input of the buffer amplifier 86. The output of the impedance converter 8 is applied via a resistor 72, which serves for signal limitation, to a positive input 78 of the comparator 76, in order there to form a variable reference for the zero threshold.

With this circuit, the reference for detection of the zero crossing as the generator supply rises is shifted upwards by a small amount (about 3% of the voltage in the DC link in the exemplary embodiment illustrated). This reduces the risk of incorrect detection of the zero crossings, in particular in the presence of load-dependent decay of the generator and of critical damping. At the other end of the spectrum, however, namely when the generator voltage is very small, the zero crossings can again be detected reliably as a result of the variable reference. This is advantageous in particular in the case of very low-impedance loads, since, due to the low voltage level that now prevails, the zero crossings can still be reliably detected. This is illustrated in FIG. 5. FIG. 5b there shows operation with regular voltage, while FIG. 5a shows operation with low voltage in which the reference threshold (dashed line) is lowered with respect to that of FIG. 5b.

To increase the detection reliability further, a correction circuit 9 against a DC voltage offset in the signal line 60 is also provided at the signal line 60. In terms of the alternating voltage, the position of the tap at the capacitive voltage divider 6 is strictly defined, but this does not apply to the direct voltage potential. In order to prevent the direct voltage potential from drifting away, and thus potentially undefined states at the comparator 76 of the zero-crossing detector 7, the correction circuit is provided with a resistor 90 that connects the signal line 60 to ground through a high resistance. The values for this resistor 90, and also those for the capacitors 61, 62, are selected here in such a way that the cut-off frequency of a possible parasitic high-pass filter is low enough that there is no longer any practical influence in the frequency range of a few 100 kHz that is of interest for the high-frequency application. A pull-up resistor 79 is provided, again to avoid undefined states at the output of the zero-crossing detector 7.

Overall, significant improvements result from the exemplary embodiment according to the invention, so that even at very low output powers of up to 5 W or less, the generator oscillation does not stall, and the zero crossings of the high-frequency signal output can be detected significantly more accurately and quickly. The operational safety is also significantly improved by the design according to the invention in respect of significant load impedance jumps.

1. An electrosurgical generator designed to output a high-frequency alternating voltage to an electrosurgical instrument, comprising a power supply unit which, when operating, feeds a direct voltage circuit, and an inverter for high voltage that is fed from the direct voltage circuit and generates a high-frequency alternating voltage that is applied to outputs for connection of the electrosurgical instrument, wherein the inverter has a clock-driven power switch and a zero-crossing detector designed to detect zero crossings of the oscillation generated by the inverter, wherein
a signal for the generated alternating voltage is applied to the zero-crossing detector by means of a first voltage divider via a signal line,
wherein
the voltage divider is designed as a capacitive voltage divider for alternating voltage with at least one capacitor resistant to high voltage.

2. The electrosurgical generator as claimed in claim 1, wherein the capacitive voltage divider has a division ratio between 1:20 and 1:4.

3. The electrosurgical generator as claimed in claim 1, wherein values of the capacitors of the capacitive voltage divider lie in the range between 50 pF and 10 nF.

4. The electrosurgical generator as claimed in claim 1, wherein the capacitive voltage divider is connected in parallel with the power switch.

5. The electrosurgical generator as claimed in claim 1, wherein the capacitive voltage divider is connected to the alternating voltage generated by the power switch directly or by means of a current limiting element.

6. The electrosurgical generator as claimed in claim 5, wherein an ohmic resistor, the resistance value of which is less than the impedance of the capacitive voltage divider, is provided as the current limiting element.

7. The electrosurgical generator as claimed in claim 1, wherein the signal line comprises a correction circuit for direct voltage offset, designed to minimize or remove a direct voltage potential in the signal line, wherein the correction circuit is implemented as a high-pass filter.

8. The electrosurgical generator as claimed in claim 1, wherein a variable reference is applied as a zero reference to the zero-crossing detector.

9. The electrosurgical generator as claimed in claim 8, wherein the variable reference is derived from the voltage in the direct voltage circuit.

10. The electrosurgical generator as claimed in claim 8, wherein the variable reference is generated by means of a second voltage divider that has a different type of construction from the first voltage divider.

11. The electrosurgical generator as claimed in claim 10, wherein an impedance converter is connected between the second voltage divider and the zero-crossing detector.

12. The electrosurgical generator as claimed in claim 11, wherein an offset circuit is provided in the reference line, being designed, in the absence of an input signal, to apply a defined reference to the zero-crossing detector via the reference line.

13. The electrosurgical generator as claimed in claim 12, wherein the offset circuit is integrated into the impedance converter.

14. The electrosurgical generator as claimed in claim 8, wherein limiting circuits are provided at the input to the zero-crossing detector for the signal line and/or the reference line.
